(19) United States
(12) Patent Application Publication
(10) Pub. No.: US 2018/0200369 A1
(43) Pub. Date: Jul. 19, 2018

(54) Vitamin E tocotrienols inhibition of intracellularly obligate pathogen Chlamydia and methods of use

(71) Applicant: AMERICAN RIVER NUTRITION, INC., Hadley, MA (US)

(72) Inventors: Barrie TAN, Amherst, MA (US); Anne Mueller, Sunderland, MA (US); Elizabeth S. Stuart, Amherst, MA (US)

(21) Appl. No.: 15/412,045

(22) Filed: Jan. 22, 2017

Related U.S. Application Data
(63) Continuation of application No. 11/411,079, filed on Apr. 24, 2006, now abandoned.
(60) Provisional application No. 60/778,432, filed on Mar. 1, 2006.
(60) Provisional application No. 60/673,837, filed on Apr. 22, 2005.

(51) Int. Cl. A61K 45/06; A61K 31/355; A61K 36/185; A61K 31/045

(52) U.S. Cl. CPC A61K 45/06; A61K 31/355; A61K 36/185; A61K 2300/00; A61K 31/045

(57) ABSTRACT

This invention reveals the beneficial use of vitamin E tocotrienols for inhibition of chlamydial infections. Chlamydial infection levels in mouse macrophages treated with tocotrienol were decreased >50%, with concomitant aberrant pathogen development. The number of large and small inclusions in tocotrienol-versus-control cells was decreased 3-fold and 2-fold, respectively. When treated with delta tocotrienol, Chlamydia in human lymphocytes was inhibited by at least 2.6-fold in 1.5 days. Dietary delta tocotrienol inhibited Chlamydia infection and persistence in hypercholesterolemic patients with a corresponding drop in LDL. These studies demonstrate that tocotrienol lowers cholesterol, thus preventing or diminishing the cholesterol hijacking by Chlamydia obligatory for its infectivity and replication. Therefore, hypolipidemic agents used to treat cardiovascular diseases, metabolic syndrome, and diabetes are used as monotherapies, or in combination with tocotrienol to treat Chlamydia.

### CROSS-REFERENCE TO RELATED APPLICATIONS

This application is a Continuation of and claims priority upon U.S. application Ser. No. 11/411,079 filed on Apr. 24, 2006 (pending), which is a Non-provisional application of U.S. Provisional Patent application Ser. No. 60/673,837 filed on Apr. 22, 2005 and U.S. Provisional Patent application Ser. No. 60/778,432 filed on Mar. 1, 2006, the contents of which are all herein incorporated by this reference in their entireties. The contents of co-pending U.S. patent application Ser. No. 10/823,043 filed on Apr. 12, 2004 and U.S. patent application Ser. No. 10/821,679 filed on Apr. 8, 2004 are all herein incorporated by this reference in their entireties. All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### OTHER REFERENCES

Azenabor A A, et al.: Chlamydia pneumoniae infected macrophages exhibit enhanced plasma membrane fluidity and show increased adherence to endothelial cells. Mol Cell Biochem 2005, 269(1-2): 69-84.
Byrne G I, et al.: Chlamydia and apoptosis: life and death decisions of an intracellular pathogen. Nat Rev Microbiol. 2004, 2: 802-8.
Carabeo R A, et al.: Golgi-dependent transport of cholesterol to the Chlamydia trachomatis inclusion. Proc Natl Acad Sci USA 2003, 100(11): 6771-6.
English J: Do your antioxidants suppress enough free radicals? Life Extension 2005, February: 22-31.
English J: Novel dietary supplement shows dramatic effects in lowering cholesterol, LDL, and triglycerides. Life Extension 2005, November: 28-38.
Gabel B R, et al.: Lipid raft-mediated entry is not required for Chlamydia trachomatis infection of cultured epithelial cell. Infection and Immunity 2004, 72(12): 7367-7373.
Granato H: Cardiovascular Health. Natural Products Industry Insider 2005, January 3: 24-40.
Granato H: Cardiovascular Wellness. Natural Products Industry Insider 2005, January 6: 20-35.
Grayston J T, et al.: A new respiratory tract pathogen: Chlamydia pneumoniae strain TWAR. J Infect Dis 1990, 161(4): 618-25.
Kooyenga, et al.: Antioxidants modulate the course of carotid atherosclerosis: A four-year study. Micronutrient & Health: Molecular Biological Mechanisms. Nesaretnam K, Packer L (Eds.). AOCS Press: Illinois, 2001. p. 366-375.
Ling Z, Bundey R A: Statin treatment of human vascular endothelial cells disrupts caveolae and increases nitric oxide signaling. FASEB J 2006, 20: 787.3.
Mo H B, Elson C E: Studies of the isoprenoid-mediated inhibition of mevalonate synthesis applied to cancer chemotherapy and chemoprevention. Exp Bio Med 2004, 229: 567-585.
Myers S: How to avoid a broken heart: using nutrients to control the leading risk factors of heart disease. Insider 2006, January 9: 22-30.
Naguib Y: Natural alternatives for maintaining healthy cholesterol. Vitamin Retailer 2004, October: 58-61.
Parker R A, et al.: Tocotrienols regulate cholesterol production in mammalian cells by post-transcriptional suppression of 3-hydroxy-3-methylglutaryl-coenzyme A reductase. J Biol Chem. 1993, 268: 11230-8.
Pearce B C, et al.: Hypocholesterolemic Activity of Synthetic and Natural Tocotrienols. J. Med. Chem. 1992, 35: 3595-06.
Sawayama Y, et al.: Association of Chlamydia pneumoniae antibody with the cholesterol-lowering effect of statins. Atherosclerosis 2003, 171: 281-285.
Srejic E: Keeping the ticker in tip-top shape. HSR Health Supplement Retailer 2006, March: 20-28.
Strum S, Faloon W: Beta-Sitosterol and the aging prostate gland. Life Extension 2005, June: 27-31.
Stuart E S, et al.: Lipid rafts, caveolae, caveolin-1, and entry by Chlamydiae into host cells. Exp Cell Res. 2003, 1: 67-78.
Tan B.: Appropriate Spectrum Vitamin E and New Perspectives on Desmethyl Tocopherols and Tocotrienols. JANA 2005, 8(1): 35-42.
Yamazaki T, et al.: Biosynthesized tea polyphenols inactivate Chlamydia trachomatis in vitro. Antimicrob Agents Chemother 2005, 49(6): 2501-2503.
Yamazaki T, et al.: The inhibitory effect of antihyperlipidemic drugs on the growth of Chlamydia pneumoniae in vitro. J Chemother 2006, 18(1): 107-9.

### FIELD OF THE INVENTION

The invention is on the use of Vitamin E to inhibit and disrupt the developmental cell cycle and infection of Chlamydia, and its use to alleviate the effects of Chlamydia-related diseases.

### BACKGROUND OF THE INVENTION

Chlamydia is an obligate intracellular pathogen known to be associated with several diseases that are common today. Chlamydia trachomatis is the primary cause of bacterial sexually transmitted disease (STD), and can lead to ectopic pregnancies and infertility. Chlamydia pneumoniae causes respiratory tract infections including bronchitis, pneumonia, sinusitis, and pharyngitis. In addition, it is linked to numerous pathologies, including Alzheimer's disease, multiple sclerosis, atherosclerosis, coronary heart disease, and asthma. Three recent Chlamydia findings are 1) various strains of clinically important Chlamydia species are associated with caveolin, a molecule important to cholesterol homeostasis, 2) apoptosis (programmed cell death) is downregulated among infected cells (Byrne et al., 2004), and 3) treatment of chlamydial infections with antibiotics drives the pathogens into a resistant and persistent state. Clinical persistence is an essential element of chlamydial pathogenesis, where the inability of the host to eliminate the pathogen leads to a state of chronic infectivity along with attendant tissue injury.

Delta tocotrienol is a vitamin E compound also available as dietary supplement. It has been shown to lower cholesterol in a controlled fashion, and to induce apoptosis in cancer cells, reducing tumors by as much as 70%. Since Chlamydia species enter cells via cholesterol-rich lipid raft domains involved in cholesterol trafficking, tocotrienols in general and hypocholesterolemic delta tocotrienol in particular will reduce infection by Chlamydia. Mouse macrophages (J774A.1), human mammary tumor cells (MCF-7, TMX2-28), human epithelial cells (Hep-2), and human B-lymphocytes (JY) were incubated with delta tocotrienol at concentrations of 10-30 μmol/L for 6 hours prior to infection by C. trachomatis serovar K, a subspecies of Chlamydia that is the primary cause of bacteria-initiated STD. Infections were detected by immunofluorescence staining followed by either microscopy or quantitative flow cytometric analysis. Infection levels in cells pretreated with delta-tocotrienol were decreased by >50%, with concomitant aberrant pathogen development observed with confocal microscopy. The number of large and small inclusions in the delta tocotrienol-versus-control cells was decreased by 3- and 2-fold, respectively. Flow cytometry showed that chlamydial inhibition in JY cells was at least 2-fold for an infection period of 72 hours, with a 2.6-fold maximum inhibition at 36 hours. The impact of dietary delta tocotrienol on Chlamydia infection in hyperlipidemic patients is being examined in a clinical study. Cholesterol-suppressive delta tocotrienol may have the potential to reduce Chlamydia infection in humans.

Chlamydia is an obligate intracellular pathogen, which means that it has to invade cells first in order to successfully survive inside the host. This infection is initiated by the so-called chlamydial elementary bodies (EBs). Once inside host cells, chlamydial replication occurs within a segregated, membrane-bound compartment called an inclusion that progressively enlarges due to metabolically active Chlamydia replication within the host cell.

Species of the genus Chlamydiaceae that are frequently associated with chronic human diseases are Chlamydiophila pneumoniae and Chlamydia trachomatis. C. pneumoniae causes 5-10% of respiratory tract infections in adults and children including bronchitis, pneumonia, sinusitis, and pharyngitis. Additionally, it has been linked to chronic diseases like late onset Alzheimer's, multiple sclerosis, reactive arthritis, atherosclerosis, and asthma. Infection by C. pneumoniae has been suggested to induce autoimmunity. In multiple sclerosis (MS), 73% of the patients were shown to be Chlamydia-positive compared to 22% of controls. In addition, C. pneumoniae is involved in atherosclerotic processes such as cellular oxidation of LDL and macrophage foam cell formation. By age 20, 50% of the population exhibits evidence of past infection by C. pneumoniae (shown by serological tests), and re-infection is common. Additionally, in a study examining a normal blood donor population for chlamydial infection levels, 25% of the population was found to be positive by immunostained blood smear. Therefore, it is possible that serological tests (e.g., ELISA) overestimate immunostaining of blood tests (e.g., actual WBCs) by ≥two-fold. C. trachomatis, on the other hand, is the world's leading cause of preventable infectious blindness, and the most common cause of STD. In females, infection with C. trachomatis initially affects mucosal membranes and leads to continual inflammation of tissue in the genital tract, which results in scarring and eventual infertility. After invasion of cells, Chlamydia aggregate in vacuole-like structures called inclusions, and can escape many of the first line host defenses of the immune system. Another feature of the Chlamydia is that they prevent apoptosis of infected cells (Byrne, et al., 2004). This effect is protective for the pathogen because it can complete a full replicative cycle within the single host cell (FIG. 1). Since chlamydial inclusions inside a single infected host cell require incubation to give rise to 200-1,000 new infectious units, inhibition of host cell apoptosis by Chlamydia is advantageous to the pathogen and supports the establishment of a prolonged infection. Therefore, one aspect of the invention is that tocotrienols will inhibit chlamydial growth inside host cells by causing the infected host to undergo apoptosis.

Delta tocotrienol is a vitamin E compound. Cancer studies with this compound have shown that it induces apoptosis of the tumor cells, but does not harm the surrounding healthy cells. Learning that delta tocotrienol has this ability, at least for cancer cells, triggered the idea that the compound may also up-regulate apoptosis of cells infected by Chlamydia. Since chlamydial inhibition of host cell apoptosis is important to the pathogen's success, such an effect by delta tocotrienol could have a profound impact as treatment against Chlamydia. Another delta tocotrienol characteristic of importance is its cholesterol-lowering effect (Pearce et al., 1992). Chlamydia enters the host cell via cholesterol-rich lipid-rafts or caveolae, which are comprised of lateral assemblies of cholesterol and sphingolipids that float in the glycerophospholipid membrane, and are impaired with removal of plasma membrane cholesterol. Therefore, cells that are exposed to delta tocotrienol could remodel the lipid rafts, and thus interfere with pathogen entry into host cells directly or indirectly. Since Chlamydia is metabolically inactive outside the host, it is unable to survive outside the cell. A potential strategy is to inhibit cholesterol synthesis or availability in order to contain or eliminate Chlamydia.

In this invention, we tested delta tocotrienol's effect on chlamydial infection in numerous normal white blood cells, cancer cells, and buffy coats of Chlamydia-positive human blood samples.

### SUMMARY OF THE INVENTION

The invention relates to uses of Vitamin E to inhibit infection by Chlamydia. Additionally, the invention relates to the mechanism of action by Vitamin E, especially delta-tocotrienol, to interrupt the infection process of a Chlamydia.

In one embodiment the invention is drawn to a method of using Vitamin E tocochromanol to inhibit and disrupt the developmental cell cycle and infection of Chlamydia. In a preferred embodiment the invention is drawn to a method of using tocotrienol to inhibit and disrupt the developmental cell cycle and infection of Chlamydia. In a more preferred embodiment the invention is drawn to a method of using delta-tocotrienol to inhibit and disrupt the developmental cell cycle and infection of Chlamydia.

In one embodiment the invention is drawn to a method of using Vitamin E tocochromanol to alleviate the effects of Chlamydia-related diseases. In a preferred embodiment the invention is drawn to a method of using tocotrienol to alleviate the effects of Chlamydia-related diseases. In a more preferred embodiment the invention is drawn to a method of using delta-tocotrienol to alleviate the effects of Chlamydia-related diseases.

In one embodiment the invention is drawn to a method of using Vitamin E tocochromanol to reduce the quantity of chlamydial inclusions. In a preferred embodiment the invention is drawn to a method of using tocotrienol to reduce the quantity of chlamydial inclusions. In a more preferred embodiment the invention is drawn to a method of using delta-tocotrienol to reduce the quantity of chlamydial inclusions.

In one embodiment the invention is drawn to a method of using Vitamin E tocochromanol to impede and suppress the initial infection by chlamydial EBs of host cells. In a preferred embodiment the invention is drawn to a method of using tocotrienol to impede and suppress the initial infection by chlamydial EBs of host cells. In a more preferred embodiment the invention is drawn to a method of using delta-tocotrienol to impede and suppress the initial infection by chlamydial EBs of host cells.

In one embodiment the invention is drawn to a method of using 5 μmol/L delta-tocotrienol to inhibit chlamydial development within the host cell. In a preferred embodiment the invention is drawn to a method of using 10 μmol/L delta-tocotrienol to inhibit chlamydial development within the host cell. In a more preferred embodiment the invention is drawn to a method of using 20 μmol/L delta-tocotrienol to inhibit chlamydial development within the host cell. In a more preferred embodiment the invention is drawn to a method of using 30 μmol/L delta-tocotrienol to inhibit chlamydial development within the host cell. In a more preferred embodiment the invention is drawn to a method of using 40 μmol/L delta-tocotrienol to inhibit chlamydial development within the host cell.

In one embodiment the invention is drawn to a method of using delta-tocotrienol several times to reduce the level of infection by Chlamydia. In a preferred embodiment the invention is drawn to a method of using delta-tocotrienol 3 to 10 times to reduce the level of infection by Chlamydia. In a more preferred embodiment the invention is drawn to a method of using delta-tocotrienol more than 10 times to reduce the level of infection by Chlamydia. In a more preferred embodiment the invention is drawn to a method of using delta-tocotrienol repeatedly to reduce the level of infection by Chlamydia.

In one embodiment the invention is drawn to a method of using delta-tocotrienol several times to inhibit re-infection of the host cell by chlamydial EBs. In a preferred embodiment the invention is drawn to a method of using delta-tocotrienol 3 to 10 times to inhibit re-infection of the host cell by chlamydial EBs. In a more preferred embodiment the invention is drawn to a method of using delta-tocotrienol more than 10 times to inhibit re-infection of the host cell by chlamydial EBs. In a more preferred embodiment the invention is drawn to a method of using delta-tocotrienol repeatedly to inhibit re-infection of the host cell by chlamydial EBs.

In one embodiment the invention is drawn to a method of using tocotrienol to inhibit inclusion formation by 5%. In a preferred embodiment the invention is drawn to a method of using tocotrienol to inhibit inclusion formation by 10%. In a more preferred embodiment the invention is drawn to a method of using tocotrienol to inhibit inclusion formation by 15%. In a more preferred embodiment the invention is drawn to a method of using tocotrienol to inhibit inclusion formation by 20%. In a more preferred embodiment the invention is drawn to a method of using tocotrienol to inhibit inclusion formation by 25%. In a more preferred embodiment the invention is drawn to a method of using tocotrienol to inhibit inclusion formation by 30%. In a more preferred embodiment the invention is drawn to a method of using tocotrienol to inhibit inclusion formation by 40%. In a more preferred embodiment the invention is drawn to a method of using tocotrienol to inhibit inclusion formation by 45%. In a more preferred embodiment the invention is drawn to a method of using tocotrienol to inhibit inclusion formation by 50%. In a more preferred embodiment the invention is drawn to a method of using tocotrienol to inhibit inclusion formation by 60%. In a more preferred embodiment the invention is drawn to a method of using tocotrienol to inhibit inclusion formation by 75%. In a more preferred embodiment the invention is drawn to a method of using tocotrienol to inhibit inclusion formation by 90%.

In one embodiment the invention is drawn to a method of using Vitamin E tocochromanol. In one embodiment the invention is drawn to a method of using tocotrienol for at least one day. In a preferred embodiment the invention is drawn to a method of using tocotrienol for at least three days. In a more preferred embodiment the invention is drawn to a method of using tocotrienol for more than three days.

In one embodiment the invention is drawn to a method of using tocotrienol is effective in humans against Chlamydia as represented in circulating WBCs, including neutrophils, monocytes, lymphocytes, eosinophils, and basophils.

In one embodiment the invention is drawn to a method of using tocotrienol to reduce the cholesterol level and chlamydial infection in hyperlipidemic patients, and rendered the chlamydial infection status negative.

In one embodiment the invention is drawn to a method of using an agent that restricts cholesterol/lipids to restrict Chlamydia infection and growth.

In one embodiment the invention is drawn to a method of using tocotrienol to reduce Chlamydia infections in cancer patients.

In one embodiment the invention is drawn to a method of using tocotrienol to ward off pathogenic infections including Chlamydia via activation of antigen-presenting dendritic cells and T-lymphocytes.

In one embodiment the invention is drawn to a method of using tocotrienol to resist opportunistic chlamydial infection of hypertensive patients, and thereby reduce the patient's blood pressure.

In one embodiment the invention is drawn to a method of using tocotrienol to reduce cardiovascular risk factors associated with metabolic syndrome, and thereby reduce the risk of diabetes and concomitant or subsequent infection by Chlamydia.

In one embodiment the invention is drawn to a method of application of tocotrienol by aerosol sprays (to reach respiratory tract airways), oral ingestion via softgels, tablets or capsules (to reach vascular-organistic systems), topical creams, douches, and lotions (to reach genital sites), and topical liquid drops (to reach ocular sites).

In a preferred embodiment the invention is drawn to a method of application of tocotrienol by oral ingestion via softgels, tablets or capsules, and topical creams, douches, and lotions. In a preferred embodiment the invention is drawn to a method of application of tocotrienol by oral ingestion via softgels, tablets or capsules. In one embodiment of the invention is drawn to a method of administering delta tocotrienol in a range from 10 to 1000 mg per day. In a preferred embodiment the invention is drawn to a method of administering delta tocotrienol in a range from 20 to 500 mg per day. In a more preferred embodiment the invention is drawn to a method of administering delta tocotrienol in a range from 50 to 150 mg per day. In one embodiment of the invention is drawn to a method of treatment with delta tocotrienol of administering daily for one month. In a preferred embodiment of the invention is drawn to a method of treatment with delta tocotrienol of administering daily for six months. In a more preferred embodiment of the invention is drawn to a method of treatment with delta tocotrienol of administering daily for one year. In a more preferred embodiment of the invention is drawn to a method of treatment with delta tocotrienol of administering daily until infection and inflammation due to Chlamydia is cleared.

In one embodiment the invention is drawn to a method of using tocotrienol for respiratory tract infections by using an aerosol spray at a dosage of 1 spray per day. In a preferred embodiment the invention is drawn to a method of using tocotrienol for respiratory tract infections by using an aerosol spray at a dosage of 2 sprays per day. In a more embodiment the invention is drawn to a method of using tocotrienol for respiratory tract infections by using an aerosol spray at a dosage of 4 sprays per day.

In one embodiment the invention is drawn to a method of using tocotrienol for genital tract infections by using an aerosol spray at a dosage of 1 application per day. In a preferred embodiment the invention is drawn to a method of using tocotrienol for genital tract infections by using an aerosol spray at a dosage of 2 applications per day. In a more embodiment the invention is drawn to a method of using tocotrienol for genital tract infections by using an aerosol spray at a dosage of 4 applications per day.

In one embodiment the invention is drawn to a method of using tocotrienol to reduce inflammation of vascular-organistic systems due to chlamydial infection by oral ingestion of 1 dose per day. In a preferred embodiment the invention is drawn to a method of using tocotrienol to reduce inflammation of vascular-organistic systems due to chlamydial infection by oral ingestion of 2 doses per day. In a more preferred embodiment the invention is drawn to a method of using tocotrienol to reduce inflammation of vascular-organistic systems due to chlamydial infection by oral ingestion of 4 doses per day.

In one embodiment the invention is drawn to a method of using tocotrienol to reduce ocular infections and conjunctivitis due to chlamydial infection by applying liquid eye drops with 1 application per day. In a preferred embodiment the invention is drawn to a method of using tocotrienol to reduce ocular infections and conjunctivitis due to chlamydial infection by applying liquid eye drops with 2 applications per day. In a more preferred embodiment the invention is drawn to a method of using tocotrienol to reduce ocular infections and conjunctivitis due to chlamydial infection by applying liquid eye drops with 4 applications per day.

In one embodiment the invention is drawn to a method of using tocotrienol where the delta-to-gamma ratio of tocotrienols is 1:100 to 100:1. In a preferred embodiment the invention is drawn to a method of using tocotrienol where the delta-to-gamma ratio of tocotrienols is 1:25 to 25:1. In a more preferred embodiment the invention is drawn to a method of using tocotrienol where the delta-to-gamma ratio of tocotrienols is 1:10 to 10:1. In a more preferred embodiment the invention is drawn to a method of using tocotrienol where the delta-to-gamma ratio of tocotrienols is 1:5 to 5:1. In a more preferred embodiment the invention is drawn to a method of using tocotrienol where the delta-to-gamma ratio of tocotrienols is 1:1.

In one embodiment the invention is drawn to a method of using tocotrienol comprising a mixture of annatto extract and a natural extract that is an appropriate spectrum. In a preferred embodiment the invention is drawn to a method of using tocotrienol where more than 50% of the tocotrienols are delta-T3 and gamma-T3. In a more preferred embodiment the invention is drawn to a method of using tocotrienol where more than 50% of the tocotrienols are delta-T3. In a most preferred embodiment the invention is drawn to a method of using tocotrienol where it is tocopherol-free.

In one embodiment the invention is drawn to a method of using tocotrienol where the C5 unsubstituted tocotrienols are >60%, and tocopherols are <15%. In a preferred embodiment the invention is drawn to a method of using tocotrienol where the C5 unsubstituted tocotrienols are >70% C5 unsubstituted tocotrienols and <10% tocopherols. In a more preferred one embodiment the invention is drawn to a method of using tocotrienol where the C5 unsubstituted tocotrienols are >80% C5 unsubstituted tocotrienols and <5% tocopherols.

In one embodiment the invention is drawn to a method of using tocotrienol where the method of using tocotrienol is tocopherol-free with >98% tocotrienols, and tocotrienols are predominantly delta-T3 and gamma-T3. In a more preferred embodiment the invention is drawn to a method of using tocotrienol where the tocotrienol is tocopherol-free with >98% tocotrienols and tocotrienols are predominantly delta-T3.

In one embodiment the invention is drawn to a method of using tocotrienol comprising annatto extract where C5 unsubstituted tocols inhibit surface chemotactic bioactive materials (CBM). In a preferred embodiment the invention is drawn to a method of using tocotrienol comprising annatto extract where annatto C5 unsubstituted T3 inhibit surface chemotactic bioactive materials. In a more preferred embodiment the invention is drawn to a method of using tocotrienol comprising annatto extract where annatto C5 unsubstituted T3 inhibit surface chemotactic bioactive materials and inhibit the tether or adhesion of circulating monocytes and leucocytes onto stationary endothelia. In a more preferred embodiment the invention is drawn to a method of using tocotrienol comprising annatto extract where annatto C5 unsubstituted T3 inhibit surface chemotactic bioactive materials and inhibit the tether or adhesion of circulating monocytes and leucocytes onto stationary endothelia that cause the loss of vasculature integrity. In a more preferred embodiment the invention is drawn to a method of using tocotrienol comprising annatto extract where annatto C5 unsubstituted T3 inhibit surface chemotactic bioactive materials and inhibit the tether or adhesion of circulating monocytes and leucocytes onto stationary endothelia that cause the loss of vasculature integrity, and inhibit micro- and macro-vascular diseases, and atherosclerosis. In a more preferred embodiment the invention is drawn to a method of using tocotrienol comprising annatto extract where annatto C5 unsubstituted T3 inhibit CBM and inhibit pathological events selected from the group consisting of chemotaxis, vasoconstriction, hypercoagulation, glycoxidation and oxidized LDL by via HDL elevation.

In one embodiment the invention is drawn to a method of using tocotrienol where the tocotrienol is combined with other nutrients. In a preferred embodiment the invention is drawn to a method of using tocotrienol were the tocotrienol is combined with other nutrients, and the tocotrienol contains geranyl geraniol. In a more preferred embodiment the invention is drawn to a method of using tocotrienol where the nutrient is selected from the group consisting of phytosterols, oryzanols, policosanols, pantethine, red yeast rice (Monascus), oat bran, garlic, gugul lipids, chitosan, soy protein (e.g., oligo- and poly-peptides, hydrolysates), CoQ10, carnitine, magnesium, chromium, potassium, calcium, D-tyrosine, fibers (insoluble and soluble types, including beta-glucans), omega-3s (DHAs and EPAs, ALAs), and lecithin.

In one embodiment, the invention is drawn to a method of using geranyl geraniols and tocotrienols and it increases the de novo biosyntheses of all subsequent intermediate isoprenoid pool and distal products.

In one embodiment, the invention is drawn to method of using tocotrienol containing geranyl geraniols that anabolically increases the endogenous de novo synthesis of CoQ10 via geranyl geraniols elongation/prenylation of side chain and conversely CoQ10 catabolically increases the endogenous de novo synthesis of geranyl geraniols via CoQ 10 beta-oxidation.

In one embodiment, the invention is drawn to a method of supplementation, comprising the administering of a tocotrienol or a tocotrienol and geranyl geraniol, and reducing Chlamydia-induced blindness.

### DETAILED DESCRIPTION OF THE PREFERRED EMBODIMENT

In one embodiment the method administers a tocotrienol, where the tocotrienol contains delta-tocotrienol and gamma-tocotrienol, and where the delta-to-gamma ratio of tocotrienols is 1:100 to 100:1. In a preferred embodiment the method administers an tocotrienol, where the tocotrienol contains delta-tocotrienol and gamma-tocotrienol, and where the delta-to-gamma ratio of tocotrienols is 1:25 to 25:1. In a more preferred embodiment the method administers an tocotrienol, where the tocotrienol contains delta-tocotrienol and gamma-tocotrienol, and where the delta-to-gamma ratio of tocotrienols is 1:10 to 10:1. In a more preferred embodiment the method administers an tocotrienol, where the tocotrienol contains delta-tocotrienol and gamma-tocotrienol, and where the delta-to-gamma ratio of tocotrienols is 1:5 to 5:1. In a more preferred embodiment the method administers an tocotrienol, where the tocotrienol contains delta-tocotrienol and gamma-tocotrienol, and where the delta-to-gamma ratio of tocotrienols is 1:1.

In one embodiment a method administers a tocotrienol, where the method of using tocotrienol is a mixture of an annatto extract and a natural extract, and where the mixture has standardized low levels of tocopherols. In a preferred embodiment the method administers a tocotrienol, where the method of using tocotrienol is a mixture of an annatto extract and a natural extract, and where the standardized level of tocopherols is ≤50%. In a more preferred embodiment the method administers a tocotrienol, where the method of using tocotrienol is a mixture of an annatto extract and a natural extract, and where the standardized level of tocopherols is ≤20%. In a more preferred embodiment the method administers a tocotrienol, where the method of using tocotrienol is a mixture of an annatto extract and a natural extract, and where the standardized level of tocopherols is ≤10%. In a more preferred embodiment the method administers a tocotrienol, where the method of using tocotrienol is a mixture of an annatto extract and a natural extract, and where the standardized level of tocopherols is ≤1%. In more preferred embodiment the method administers a tocotrienols, where the natural extract is selected from the group consisting of a vegetable oil of rice bran, palm, cranberry seed, and litchi seed.

In one embodiment a method administers a mixture of annatto extract and a natural extract that is an appropriate spectrum. In a preferred embodiment the method administers a mixture of annatto extract and a natural extract, and more than 50% of the tocotrienols are delta-T3 and gamma-T3. In a more preferred embodiment the method administers a mixture of annatto extract and a natural extract, and more than 50% of the tocotrienols are delta-T3. In a most preferred embodiment the method administers a mixture of annatto extract and a natural extract, and it is tocopherol-free.

In one embodiment a method administers an >60% C5 unsubstituted tocotrienols and <15% tocopherols. In a preferred embodiment a method administers an >70% C5 unsubstituted tocotrienols and <10% tocopherols. In a more preferred one embodiment a method administers an >80% C5 unsubstituted tocotrienols and <5% tocopherols.

In one embodiment a method administers an annatto extract and the method of using tocotrienol is tocopherol-free with >98% tocotrienols, and tocotrienols are predominantly delta-T3 and gamma-T3. In a more preferred embodiment the method administers an annatto extract and is tocopherol-free with >98% tocotrienols and tocotrienols are predominantly delta-T3.

In one embodiment a method administers an C5 unsubstituted tocols inhibit surface chemotactic bioactive materials. In a more preferred embodiment a method administers an C5 unsubstituted tocols, where the C5 unsubstituted tocols are C5 unsubstituted T3 and the C5 unsubstituted T3 inhibit surface chemotactic bioactive materials. In a more preferred embodiment a method administers an C5 unsubstituted tocols, where the C5 unsubstituted tocols are C5 unsubstituted T3 and the C5 unsubstituted T3 inhibit surface chemotactic bioactive materials and inhibit the tether or adhesion of circulating monocytes and leucocytes onto stationary endothelia. In a more preferred embodiment a method administers an C5 unsubstituted tocols, where the C5 unsubstituted tocols are C5 unsubstituted T3 and the C5 unsubstituted T3 inhibit surface chemotactic bioactive materials and inhibit the tether or adhesion of circulating monocytes and leucocytes onto stationary endothelia that cause the loss of vasculature integrity. In a more preferred embodiment a method administers an C5 unsubstituted tocols, where the C5 unsubstituted tocols are C5 unsubstituted T3 and the C5 unsubstituted T3 inhibit surface chemotactic bioactive materials and inhibit the tether or adhesion of circulating monocytes and leucocytes onto stationary endothelia that cause the loss of vasculature integrity, and inhibit micro- and macro-vascular diseases, and atherosclerosis. In a more preferred embodiment a method administers an C5 unsubstituted tocols, where the C5 unsubstituted tocols are C5 unsubstituted T3 and the C5 unsubstituted T3 inhibit surface chemotactic bioactive materials and inhibit pathological events selected from the group consisting of chemotaxis, vasoconstriction, hypercoagulation, glycoxidation and oxidized LDL by via HDL elevation.

In one embodiment a method administers an annatto extract and the annatto extract is combined with other nutrients. In a preferred embodiment a method administers an annatto extract and the annatto extract is combined with other nutrients, and the annatto extract contains tocotrienol and geranyl geraniol. In a more preferred embodiment a method administers an annatto extracts and where the nutrient is selected from the group consisting of phytosterols, oryzanols, policosanols, pantethine, red yeast rice (Monascus), oat bran, garlic, gugul lipids, chitosan, soy protein (e.g., oligo- and poly-peptides, hydrolysates), CoQ10, carnitine, magnesium, chromium, potassium, calcium, D-tyrosine, fibers (insoluble and soluble types, including beta-glucans), omega-3s (DHAs and EPAs, ALAs), and lecithin. In another embodiment a method administers an annatto extract and a nutrient, and the nutrient is selected from the group consisting of banaba extract (e.g., corosolic acid), lipoic acids (all isomeric forms), chromium, and the B vitamins including niacin.

In one embodiment, a method administers an geranyl geraniols and tocopherol-free C-5 unsubstituted tocotrienols. In a more preferred embodiment, the method administers an geranyl geraniols, tocopherol-free C-5 unsubstituted tocotrienols, and inactive and/or active ingredients.

In one embodiment, a method of using tocotrienol containing geranyl geraniols treats a disease of the nervous system. In a preferred embodiment, the method of using tocotrienol containing geranyl geraniols treats a disease of the nervous system, where the disease is selected from the group consisting of chronic Alzheimer's, Parkinson's, Familial Dysautonomia, Muscular Sclerosis, and Muscular Atrophy.

Some embodiments of the present invention are described with reference to the numbered paragraphs below:

1. A method of treating an infection by Chlamydia, comprising administering a Vitamin E tocochromanol to a mammal in need of treatment.

2. The method of paragraph 1, where the treatment inhibits the developmental cell cycle and infection of Chlamydia.

3. The method of paragraph 1, where the treatment disrupts the developmental cell cycle and infection of Chlamydia.

4. The method of paragraph 1, further comprising administering a geranyl geraniol.

5. The method of paragraph 1, where the mammal has a condition selected from the group consisting of elevated intracellular calcium, increased caveolae expression, increased vasoconstriction, hypertension and primary pulmonary hypertension.

6. The method of paragraph 1, where the Vitamin E tocochromanol is selected from the group consisting of a natural tocopherol, a synthetic tocopherol, a natural tocotrienol and a synthetic tocotrienol.

7. The method of paragraph 7, where the tocotrienol is an isomer of a tocotrienol.

8. The method of paragraph 8, where the isomer of tocotrienol is selected from the group consisting of alpha, beta, gamma, delta, desmethyl, and didesmethyl.

9. The method of paragraph 7, where the tocopherol is an isomer of a tocopherol.

10. The method of paragraph 10, where the isomer of tocopherol is selected from the group consisting of alpha, beta, gamma, delta, desmethyl, and didesmethyl.

11. The method of paragraph 1, where the Chlamydia is selected from the group consisting of Chlamydia trachomatis, Chlamydia suis, Chlamydia muridarum, Chlamydiophila pneumoniae, Chlamydiophila psittaci, Chlamydiophila pecorum, Chlamydiophila abortis, Chlamydiophila felis, and Chlamydiophila caviae.

12. The method of paragraph 1, where the tocotrienol stunts the growth and inhibits Chlamydia maturation of reticulate bodies into elementary bodies, and inhibits their progression and development.

13. The method of paragraph 1, where the tocotrienol reduces chlamydial infection of a white blood cell.

14. The method of paragraph 14, where the white cell is selected from the group consisting of neutrophils, monocytes, lymphocytes, eosinophils, and basophils.

15. The method of paragraph 7, where the tocotrienol lowers cholesterol in hypercholesterolemic patients to inhibit chlamydial infection.

16. A method of treating an infection by Chlamydia, comprising administering an agent that restricts cholesterol.

17. The method of paragraph 17, where the agent that restricts cholesterol is selected from the group consisting of a statin, a bioflavonoid, a polyphenolic, a polymethoxylated flavone, a plant sterol, a oryzanol, a policosanol, a B vitamin, CoQ10, an omega 3 fatty acid, a lecithin, garlic, a gugul lipids, an insoluble fiber, a soluble fiber, a soy protein, a chitosan, a red yeast rice, and a mineral.

18. The method of paragraph 18, where the bioflavonoid is selected from the group consisting of citrus bioflavonoid and polymethoxylated flavone.

19. The method of paragraph 1, where mode of application of the Vitamin E tocochromanol is selected from the group consisting of aerosol spray, oral ingestion, creams, douches, lotions, and eye drops.

20. The method of 20, comprising administering a dose of tocotrienol between 10 mg and 1000 mg per day.

21. The method of 21, comprising administering a dose of tocotrienol between 20 mg and 500 mg per day.

22. The method of 22, comprising administering a dose of tocotrienol between 50 mg and 150 mg per day.

23. A method of treating an infection by Chlamydia, comprising administering a combination of a tocotrienol and at least one agent selected from the group consisting of a statin, a bioflavonoid, a polyphenolic, a polymethoxylated flavone, a plant sterol, a oryzanol, a policosanol, a B vitamin, CoQ10, an omega 3 fatty acid, a lecithin, garlic, a gugul lipids, an insoluble fiber, a soluble fiber, a soy protein, a chitosan, a red yeast rice, and a mineral.

24. A method of treating an infection by Chlamydia, comprising administering a combination of a polymethoxylatyed flavone and at least one agent selected from the group consisting of a tocotrienol, a statin, a bioflavonoid, a polyphenolic, a polymethoxylated flavone, a plant sterol, a oryzanol, a policosanol, a B vitamin, CoQ10, an omega 3 fatty acid, a lecithin, garlic, a gugul lipids, an insoluble fiber, a soluble fiber, a soy protein, a chitosan, a red yeast rice, and a mineral.

25. A method of treating Chlamydia-associated diseases, comprising administering a hypocholesterolemic agent to a mammal or avian to treat a Chlamydia-associated disease selected from the group consisting of cardiovascular disease, hypertension, atherosclerosis, COX-I- and COX-II-induced inflammation, a sexually transmitted disease, a genital tract infection, arthritis, prediabetes, metabolic syndrome, diabetes, polycystic ovarian syndrome (PCOS), a respiratory tract infection, pneumonia, an ocular infection, a neurological disease, Alzheimer's disease, and multiple sclerosis.

26. The method of Paragraph 26, where the hypocholesterolemic agent is a tocotrienol.

27. The method of Paragraph 27, where tocotrienol inhibits intracellular calcium, [Ca²⁺], and caveolae expression, and thereby reduces vasoconstriction and primary pulmonary hypertension (PPH), and therefore inhibits chlamydial infection associated with hypertension.

28. A method to improve immunity to Chlamydia, comprising administering a tocotrienol to a mammal to potentiate an antigen-presenting dendritic cell and improve immunity against Chlamydia.

29. A method to inhibit progression of a cancer, comprising administering a tocotrienol to a mammal to potentiate an antigen-presenting dendritic cell and inhibit progression of a cancer.

### Other Embodiments

It is to be understood that while the invention has been described in conjunction with the detailed description thereof, the foregoing description is intended to illustrate and not limit the scope of the invention, which is defined by the scope of the appended claims. For example, although the above description relates to human cells, various aspects of the invention might also be applied to cells from other animals (e.g., mammals, avians, fish, crustaceans, and domestic and farm animals) by making appropriate modifications to the described methods. Other aspects, advantages, and modifications are within the scope of the following claims.

### BRIEF DESCRIPTION OF THE SEVERAL VIEWS OF THE DRAWING

FIG. 1 illustrates the Chlamydial Developmental Cycle. In step (1), elementary bodies (EBs) infect host cells by a process similar to receptor-mediated endocytosis, and form a vacuole-like structure called an inclusion (2). The EBs then transform into non-infectious reticulate bodies (RBs), in which state they replicate and push the nucleus to the side of the cell (2→3). The inclusion enlarges, the RBs transform back to EBs (3), and the inclusion eventually lyses the cell (4). The freed infectious EBs are now able to re-infect surrounding host cells.

FIG. 2 illustrates the Molecular and Chemical Structure of Delta Tocotrienol. Delta tocotrienol is a Vitamin E compound with a chromanol nucleus (site of antioxidant activity, typical for all Vitamin E compounds), and an isoprenoid tail (farnesyl tail). The farnesylated tail downregulates the rate-limiting cholesterol biosynthesis enzyme, 3-hydroxy-3-methylglutaryl coenzyme A (HMG-CoA) reductase (Parker et al., 1993). This cholesterol inhibition by delta tocotrienol is unique to farnesylated Vitamin E compounds. Among the known isomers of tocotrienol are alpha, beta, gamma, and delta tocotrienol. The potency of cholesterol inhibition by these isomers is delta>gamma>beta>alpha tocotrienol. Apparently, desmethyl tocotrienols are more active, especially in the absence of a methyl group at C5 on the benzene ring (see arrow). Delta tocotrienol is monomethylated at position 8 of the benzene ring, making it the least substituted, and therefore the most potent isomer of the four common tocotrienol compounds (Tan, 2005).

FIG. 3 illustrates Chlamydia-infected mouse macrophage cells treated with delta tocotrienol (400× magnification). Mouse macrophages (J774A.1 cells) were pretreated with a 30 μmol/L concentration of delta tocotrienol in culture for 4 hours and infected with C. trachomatis SVK for 48 hours. Arrows indicate inclusions. Cells were viewed and digitally documented using a Zeiss LSM 510 Meta Confocal System at a magnification of 400×. White arrows point out characteristic inclusions (Scale indicates 50 μm).

FIG. 4 illustrates Chlamydia-infected mouse macrophage cells treated with delta tocotrienol (630× magnification). White arrows point to representative cells. Note numerous very small, unfused inclusions in A (Scale indicates 50 μm).

FIG. 5 illustrates flow cytometry on Chlamydia-infected human B-lymphocytes treated with delta tocotrienol. Human B-lymphocyte (JY) cells were treated with a 30 μmol/L concentration of delta tocotrienol in culture for 4 hours, infected with C. trachomatis SVK for 24-72 hours. Immunofluorescent Chlamydia-positive cells were detected by flow cytometry.

FIG. 6 illustrates detection and quantification of Chlamydia-infected white blood cells in hyperlipidemic patients with delta-tocotrienol supplementation. Buffy coats of hyperlipidemic patients were immunostained, and for each sample, aliquots of 10,000 cells were assessed. A dotplot of a hyperlipidemic patient prior to supplementation with delta-tocotrienol is shown in the left panel, and a dotplot of a hyperlipidemic patient after 2 months of delta-tocotrienol supplementation (100 mg/day) is shown in the right panel.

FIG. 7 illustrates Chlamydia-infected human mammary tumor cells treated with delta tocotrienol (630× magnification). Human mammary tumor cells (TMX2-28) were infected with Chlamydia for 72 hours and treated with a 15 μmol/L concentration of delta tocotrienol (FIG. 7A). Controls were left untreated (FIG. 7B).

### DETAILED DESCRIPTION OF THE INVENTION

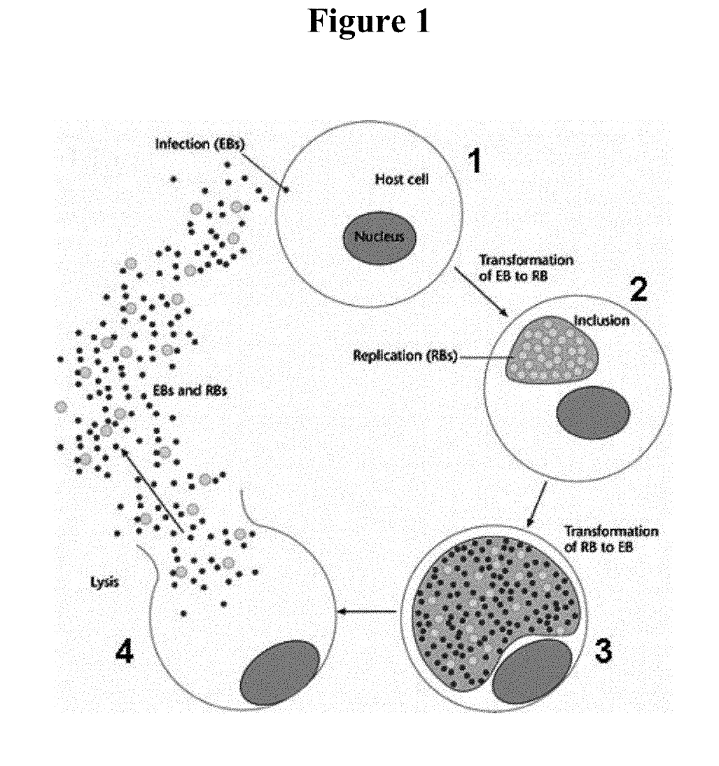

The developmental cycle of Chlamydia consists of the infectious, but metabolically inactive elementary body (EB) that will initiate cell entry, and the metabolically active, but non-infectious reticulate body (RB), which replicates within the cell and differentiates back to EBs prior to release from the infected cell [FIG. 1].

Microbes usurp normal host cell endocytic pathways to gain entry. Several infectious agents, including viruses and intracellular parasites, were found to enter host cells via caveolae or rafts. Many mechanisms for the entry of Chlamydia into host cells are proposed. These include the passage-like caveolae, cavity-like clathrin, and/or other pathways. Since only the EB form of Chlamydia is infectious and the EB at 300 nm is about 3-fold larger than either caveolae or clathrin (both ca. 100 nm), it seems unlikely that Chlamydia would enter host cells via these mechanisms. Chlamydia entry into host cells and subsequent infection requires lipid rafts, especially those that are rich in cholesterol content. Severe sequestration of cholesterol by cholesterol precipitation/chelation (Stuart et al., 2003), as well as, inhibition of cholesterol synthesis using antihyperlipidemic drugs (Yamazaki et al., 2006) interferes with host cell endocytosis of Chlamydia. Therefore, Chlamydia is more likely to enter cells via lipid rafts than some form of caveolar enclosure (invagination). In addition, statins were found to disrupt caveolae in human vascular endothelial cells, thus providing an alternate route of Chlamydia inhibition (Ling and Bundey, 2006), hitherto unknown. Such route of Chlamydia inhibition may further be supported by reduction of cholesterol with statin. The raft-mediated endocytosis may utilize a zipper-type process, whereby the lipids fuse or coalesce onto the EB to facilitate entry. Such fusion of cholesterol materials onto the EB is plausible, since Chlamydia cannot synthesize its own lipids, and is known to obtain cholesterol from its host. This cholesterol from the host may be from de novo synthesis in the host cell or from the systemic circulation ex vivo where it was transported from the liver. Chlamydia obtains cholesterol preferentially from extracellular sources by trafficking from the Golgi apparatus, which is then found in large amounts in the chlamydial inclusion membrane (Carabeo et al., 2003). De novo-synthesized cholesterol is mediated via lipid-rich transport intermediates by energy- and temperature-dependent transport to the plasma membrane.

In a 2-year clinical study on hyperlipidemic patients positive for C. pneumoniae, the cholesterol-lowering drug pravastatin did not induce a significant decrease in serum total- and LDL-cholesterol (1-2% drop), but reduced carotid atherosclerosis by 12%, significantly (Sawayama et al., 2003). This study is instructive in that if these patients were not Chlamydia-positive, cholesterol levels might have decreased significantly by administration of pravastatin, and carotid atherosclerosis might have been reduced by more than 12%. Another study found that Chlamydia-infected cells showed an enhanced uptake of LDL in macrophages, thereby contributing to foam cell and eventual atherogenic lesion formation. Put together, these studies imply that a relationship exists between Chlamydia infection, cholesterol, and carotid atherosclerosis. Earlier pathological studies have shown a positive association between prior or current infection with C. pneumoniae and coronary artery disease (Grayston et al., 1990). Interestingly, in a 4-year study, tocotrienol caused a regression of human carotid atherosclerosis without a significant cholesterol drop (Kooyenga et al., 2001).

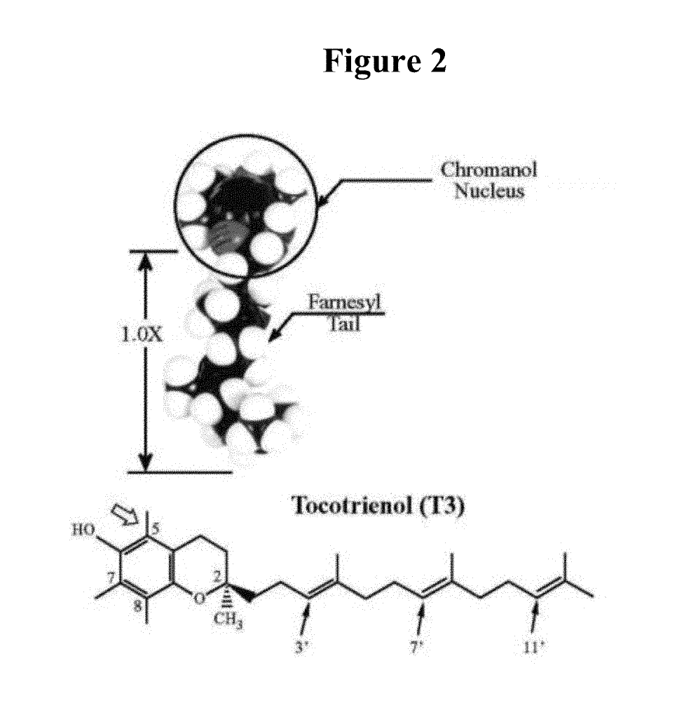

Tocotrienols [FIG. 2] belong to the same group of vitamin E as tocopherols, and their ring structure gives these compounds antioxidant properties. Tocotrienol specifically inhibits de novo synthesis of cholesterol via the HMG-CoA reductase pathway. It is expected that the tocotrienol, while lowering systemic LDL, will compromise the constitution of the cholesterol-rich lipid rafts in host cell outer membranes. Importantly, tocotrienol inhibits monocyte-endothelial cell adhesion, which in turn aids a more uniform distribution in the membrane bilayer, and greatly reduces the risk of developing atherosclerotic lesions. Therefore, it is further expected that tocotrienol inhibits the infection of pathogen to host by adhesion of Chlamydia to the host.

Once chlamydial entry into host cells has occurred, the bacteria have an ongoing requirement for cholesterol to continue pathogen development. This cholesterol is specifically derived from the membrane of host cells (Azenabor, 2005), and it has been shown that this process is mediated via the Golgi organelles (Carabeo et al., 2003). The depletion of cholesterol from host cell membrane occurs because the Chlamydia inclusion hijacks cholesterol the host cell produces, making it unavailable for the host cell membranes. This results in membrane fluidity, increased adherence of macrophages to endothelial cells, and subsequently the risk of developing atherogenic lesions. Tocotrienol is expected to inhibit intracellular synthesis of cholesterol, thus reducing cholesterol hijacking and Golgi trafficking of cholesterol by Chlamydia. Therefore, tocotrienol is expected to inhibit the progression or development of Chlamydia from RB to EB, and concomitantly limits the enlargement process of chlamydial inclusion formation. Finally, this would reduce or retard bacterial replication in infected cells that support subsequent infection of uninfected host cells, which occurs following lysis of Chlamydia-infected host cells [FIG. 1, steps 4→1]. It also may be possible that lipid raft-mediated chlamydial entry is not required for infection (Gabel et al., 2004). Therefore, tocotrienol may inhibit chlamydial infection in mechanism(s) besides the cholesterol reduction route. Nonetheless, tocotrienol inhibits chlamydial infection. However, the cholesterol reduction route may still account for the inhibition of chlamydial progression.

Therefore, the summary of this invention is that delta tocotrienol inhibits cholesterol de novo synthesis, disrupting or inhibiting lipid raft formation, although its involvement in restricting lipid rafts may not be necessary. By this invention, tocotrienol restricts or halts the entry (endocytosis) via pathogen-host adhesion and the progression of the various stages of the chlamydial developmental cycle. Delta tocotrienol may have a dual purpose of simultaneous inhibition of infection and arrest of normal chlamydial developmental progression that would lead to formation of new infectious EBs. Therefore, this invention describes that the inhibition of cholesterol in the cholesterol-rich lipid raft impedes the progression of Chlamydia infection in all of its developmental stages.

### Definitions

Assorted Nutritional Supplements—Plant sterols, oryzanols, corosolic acid, policosanols, B vitamins (e.g., pentathine, niacin, carnitine, alpha-lipoic acid, taurine), CoQ10, omega 3 fatty acids (e.g., DHAs, EPAs, alpha linoleic acid), lecithins (e.g., phosphotidyl-choline, serine, ethanolamine, inositol), garlic, gugul lipids, insoluble and soluble fibers, soy protein (e.g., oligo- and poly-peptides, hydrolysates), chitosan, red yeast rice (Monascus), and minerals (e.g., magnesium, calcium, chromium, potassium).

Caveolae—Small pockets, vesicles, caves, or recesses communicating with the outside of a cell and extending inward, causing indents in the cytoplasm and cell membrane. Such caveolae may be pinched off to form free vesicles within the cytoplasm. They are considered sites for uptake of materials into the cell, and are one of the routes Chlamydia takes to enter host cells. Caveolae are also sites of expulsion of materials from the cell, or sites of addition or removal of cell (unit) membrane to or from the cell surface.

Chlamydia—Bacteria belonging to the order of Chlamydiales, and are contained in the family of obligate intracellular organisms, which includes other obligate intracellular bacteria, viruses, and parasites, as well as the different species of Chlamydia such as Chlamydia trachomatis, Chlamydiophila pneumoniae, Chlamydiophila pecorum, Chlamydiophila psitacci, Chlamydiophila abortus, Chlamydiophila felis, Chlamydiophila caviae, Chlamydia suis, and Chlamydia muridarum.

Chlamydia Infection—Initial infection of host cells by Chlamydia. This occurs with the infectious morphological form of Chlamydia, the elementary body (see FIG. 1).

Chlamydia Progression—Development of Chlamydia within host cells. This involves transformation of the infectious elementary body to the non-infectious reticulate body, transformation back to the infectious elementary body form, and growth of the Chlamydia-containing inclusion up to the point of cell lysis (see FIG. 1).

Chlamydia-Reinfection—At completion of the chlamydial developmental cycle, the infected host cell undergoes cell lysis, setting free infectious elementary bodies (see FIG. 1). These infectious elementary bodies then infect neighboring host cell.

Chlamydia-Related Diseases—Cardiovascular disease, hypertension, atherosclerosis, COX-I- and COX-II-induced inflammation, sexually transmitted disease and genital tract infection, arthritis, respiratory tract infection and pneumonia, ocular infection, neurological diseases, including Alzheimer's disease and multiple sclerosis.

Clathrin-Coated Pits—Involved in internalization of receptor-bound ligands by receptor-mediated endocytosis. This is one of the pathways Chlamydia uses to enter a cell.

Cholesterol-Reducing Drugs—Statins (e.g., lovastatin, simvastatin, pravastatin), citrus bioflavonoids, or specifically polymethoxylated flavones (e.g., tangeretin, nobiletin, hesperidin, rutin), polyphenolics (e.g., EGCG, catechins, resveratrol).

Lipid Rafts—Domains high in sphingolipids and cholesterol in the cell membrane. These domains are detergent-insoluble glycolipid-rich domains and move within the fluid bilayer. These lipid rafts are one of the pathways by which Chlamydia enter host cells.

Tocochromanol—Vitamin E. This includes all individual isomers of tocopherol and tocotrienol, tocotrienol-rich fractions from natural sources such as palm, rice, and annatto, and various spectrum vitamin E (e.g., full and appropriate spectra).

### EXAMPLES

### Example 1

Chlamydial Strains:

Stocks of C. trachomatis serovar K/VR887 were grown in J774A.1 cells without centrifuge assistance. Infected cells were lysed, this stock aliquoted and frozen down in SPG freeze medium (75.0 g sucrose, 0.52 g potassium phosphate, 1.22 g sodium phosphate dibasic, 0.72 g glutamic acid, diluted in 100 ml ddH₂O). These aliquots were stored in liquid N₂ or at −80° C., and later thawed for use to infect monolayers.

Cell Lines Used:

Mouse macrophages (J774A.1), human mammary tumor cells (MCF-7), and human epithelial cells (Hep-2), were obtained from the American Type Culture collection. Human mammary tumor cells (TMX2-28) were a kind gift from Dr. Arcaro, and human B-lymphocytes (JY) were a kind gift from Dr. Eric Martz. All cell lines were maintained in Richter's improved MEM insulin (IMEMZO, Irvine Scientific, Santa Ana, Calif.) with 5% fetal bovine serum (FBS, Atlanta Biologicals, Norcross, Ga.). Cells were grown to 80% confluence on 12 mm coverslips in 12 well plates (Becton Dickinson Labware, Franklin Lakes, N.J.). A dilution of 1:125 or 1:200 of the C. trachomatis serovar K stock was made using the standard complete cycloheximide overlay media (Bio-Whittaker, Walkersville, Md.) containing 10% FBS, and 1× L-glutamine (CCOM). This was layered onto the coverslip containing monolayers, and incubated for 24-48 hours at 37° C. with 5% CO₂. Coverslips with the cell monolayers were harvested, rinsed with phosphate buffered saline (PBS), fixed with 70% cold methanol, stored and subsequently immunostained.

Delta Tocotrienol Treatment:

A stock of delta tocotrienol (98% purity, American River Nutrition, Hadley, Mass.) was diluted to 1 mg/1 ml in absolute ethanol [EtOH]. Confluent monolayers of J774A.1, MCF-7, TMX2-28, JY, or Hep-2 cells were treated with 5, 10, 20, 30, or 40 μmol/L concentrations of delta tocotrienol. Treatment occurred 5 hours prior to infection with C. trachomatis serovar K, at point of infection, or three hours post-infection.

Immunostaining:

Briefly, infected cells were immunostained with a 1:2 dilution of guinea pig anti-chlamydia polyclonal antibody (Biomeda, Foster City, Calif.), or a 1:125 dilution of rabbit anti-chlamydia EB whole serum for 1 hour at 37° C. Following PBS rinses ×3, the bound antibodies were detected using a 1:100 dilution of FITC-conjugated donkey anti-guinea pig or a 1:125 dilution of TRITC-conjugated goat anti-rabbit secondary antibodies (Jackson ImmunoResearch, West Grove, Pa.). Following incubation for 1 hour at room temperature and 3 rinses with PBS, coverslips were mounted onto slides using Fluoromount-G (Southern Biotechnology Associates Inc., Birmingham, Ala.) or Vectashield® Mounting Medium with DAPI (Vector Laboratories, Inc., Burlingame, Calif.), and were then sealed. Initially, slides were examined at 400× using a Nikon LABPHOT-2. For photography, slides were assessed in the Massachusetts Central Microscopy Facility using a Zeiss LSM 510 Meta Confocal System and 630× magnification. Images were captured, and as relevant, merged using the Confocal Assistant™ version 4.02 Image Processing Software.

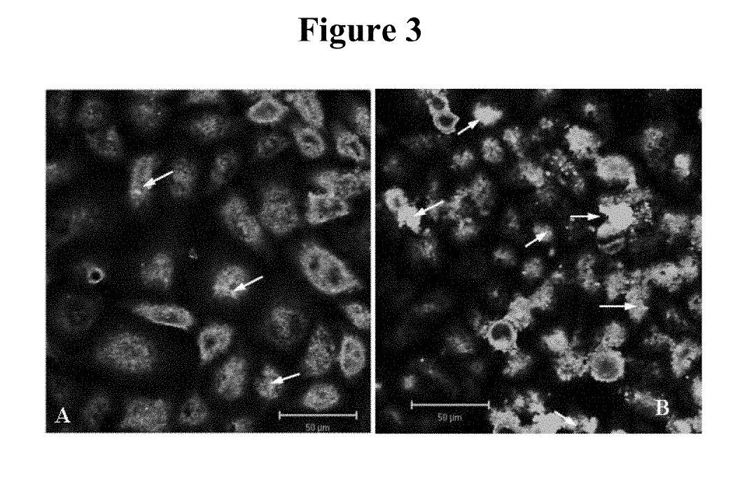

At this magnification, the differences in the number of inclusions present in the tocotrienol-treated sample (FIG. 3A) and control (FIG. 3B) were readily visualized. As the white arrows point out, no evidence of large, mature inclusions was found with tocotrienol-treatment, whereas untreated cells (3B) exemplify a high level of infection by Chlamydia, as shown by the large number and size of the inclusions present.

Quantitatively, immunofluorescence staining showed that the number of chlamydial inclusions was decreased significantly in tocotrienol-treated cells (FIG. 3, Table 1). This showed that the initial infection by chlamydial EBs of host cells was impeded and suppressed (FIG. 1, step 1).

### Example 2

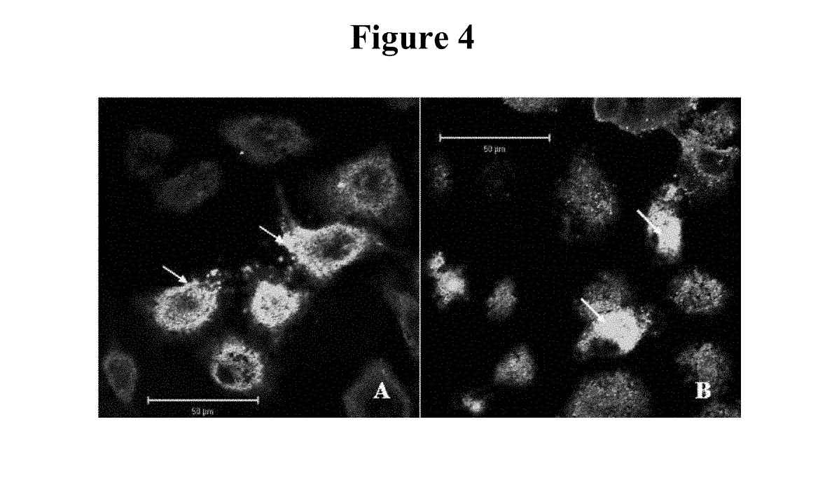

The methods of Example 1 were used in this study. Cells in culture were treated with delta tocotrienol concentrations of 5, 10, 20, 30, and 40 μmol/L, where the viscous Vitamin E compound is diluted in 100% EtOH without cytotoxicity. Controls incubated with the corresponding amounts of EtOH, as well as controls incubated with delta tocotrienol diluted in EtOH showed no difference in cell number when compared to cells grown in culture medium. At 630× magnification, the difference in size and morphology of inclusions was seen when comparing the tocotrienol-treated sample (FIG. 4A) to the control (FIG. 4B). Inclusions in (4A) were small, and did not fuse to the morphology of mature inclusions. In (4B), inclusions were mature, large, and solidly stained. Optical sections through the Z-axis (third dimension) demonstrate that these untreated cells (4B) were three-fold thicker than the tocotrienol-treated cells (4A). Therefore, the chlamydial inclusion volumes would be expected to be even larger (see Example 3).

Chlamydial inclusions in cells that were treated with higher concentrations (20-40 μmol/L) of delta tocotrienol were immature, small, and less round when visualized by Chlamydia-specific immunofluorescence staining (FIG. 4). This observation showed that chlamydial development within the host cell was grossly inhibited (FIG. 1, step 2→3).

### Example 3

TABLE 1
Summary of Inclusion and Mouse Macrophage Cell Count

| Inclusions | | | | | | |
|---|---|---|---|---|---|---|
| (per field | First Infection | | Second Infection | | Third Infection | |
| of view) | Treated | Control | Treated | Control | Treated | Control |
| Large | 0.7 | 2.6 | 1.4 | 2.6 | 0.7 | 1.2 |
| (15-20 μm) | | | | | | |
| Small | 4.5 | 10.0 | 0.6 | 3.2 | 1.1 | 2.1 |
| (≤10 μm) | | | | | | |
| Total | 5.2 | 12.6 | 2.0 | 5.8 | 1.8 | 3.3 |
| Total/Cell | 0.058 | 0.111 | 0.032 | 0.073 | 0.016 | 0.041 |

| | | | |
|---|---|---|---|
| Percent | 52.3% | 43.8% | 39.0% |
| Inhibition | | | |

* Controls were cells that were infected, but never treated with delta tocotrienol

The methods of Example 1 were used in this study. Counts on all coverslips with experimental conditions as in FIGS. 3 and 4 were done for the average of 10 fields of view. Cells were treated with 30 μmol/L concentrations of delta tocotrienol. Re-infectability was studied, where transfer of the supernatant containing infectious elementary bodies (EBs) from infected, tocotrienol-treated cells to uninfected, tocotrienol-treated cells (Table 1).

This observation suggested that repeated use of delta tocotrienol significantly reduces the level of infection by Chlamydia, and showed that delta tocotrienol inhibited chlamydial EBs from re-infecting host cells (FIG. 1, step 4→1). Therefore, tocotrienol helped the host cells to resist the repeated attempts of infection by Chlamydia.

The percent inhibition of inclusion formation in tocotrienol-treated as compared to untreated cells was approximately 40-50% (Table 1). It also should be noted that large inclusions of the control cells were larger (20 μm; more like FIG. 1, step 2→3) than large inclusions in the tocotrienol-treated cells (10-15 μm; more like FIG. 1, step 1→2). For example, tocotrienol-treated chlamydial inclusions were typically 2 to 3-fold smaller (X-Y axes) and 3-fold thinner (Z-axis; Example 2) than inclusions in control cells. The chlamydial inclusion volume thus calculated (2×3 to 3×3) was 6-9-fold smaller; suggesting that tocotrienol effectively inhibited the normal chlamydial cell cycle (FIG. 1, steps 1→2 and 2→3).

The overall effect of delta tocotrienol on Chlamydia-infected cells was that it reduced infection by influencing the pathogen's infectivity and by inhibiting its normal development (Table 1, FIGS. 3 and 4), thus compromising the pathogen's entire developmental cycle (FIG. 1). This study showed that repeated doses of tocotrienol combat Chlamydia infection and progression.

It is likely that control and eradication of Chlamydia in humans will require tocotrienol supplementation for an extended period. In light of this, tocotrienols and other agents (such as tangeretin, nobiletin, EGCG, and resveratol; more in Example 7) in monotherapies or in combination are superior because of their lack of toxicity. In contrast, other drugs (such as statins) have sustained toxicities with chronic usage. This invention highlights the use of safe natural products for fighting infections.

### Example 4

The methods of Example 1 were used in this study. Samples were analyzed for carriage of Chlamydia-infected cells by FACScan (Becton, Dickinson and Company, Franklin Lakes, N.J.).

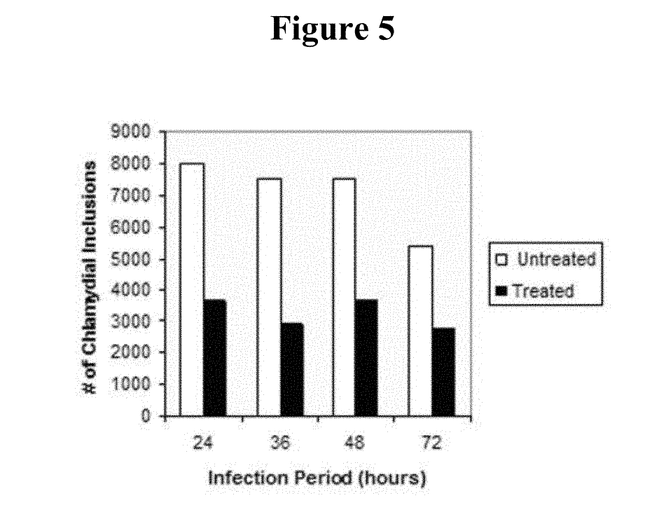

For each period, infection was decreased with delta-tocotrienol treatment. Chlamydial inhibition in JY cells was at least 2-fold over an infection period of 72 hours, with a 2.6-fold maximum inhibition at 36 hours (FIG. 5).

Clearly, chlamydial inhibition was effective in as short a period as 1 day, and following one single tocotrienol treatment, the effect was persistent for at least 3 days. This study provides showed that at least one-time dose of tocotrienol is effective against Chlamydia.

### Example 5

Flow Cytometry Assessment of WBC:

Flow cytometry (FC) quantification of Chlamydia-infected peripheral WBC used PBS rinsed buffy coat (BC) from HP samples. The BC cells were fixed and permeabilized (1% paraformaldehyde and 1% Triton X-100, 10 min. at RT; Aldrich Chemical Company, Inc., Milwaukee, Wis.). BCs were separately incubated for 1 hour with a 1:200 dilution of rabbit anti-Chlamydia primary antibody (Biodesign International, Saco, Me.) followed by a 1 hour incubation with a 1:150 dilution of FITC-conjugated goat anti-rabbit IgG (H+L) (Jackson ImmunoResearch, West Grove, Pa.), PBS rinsed ×3, and mono-dispersed by passage through a nylon mesh filter (Lab-Line Instruments Inc, Melrose Park, Ill.). Sufficient WBCs were added to obtain 10,000 cells/tube and samples were analyzed for carriage of Chlamydia-infected cells by FACScan (Becton, Dickinson and Company, Franklin Lakes, N.J.).

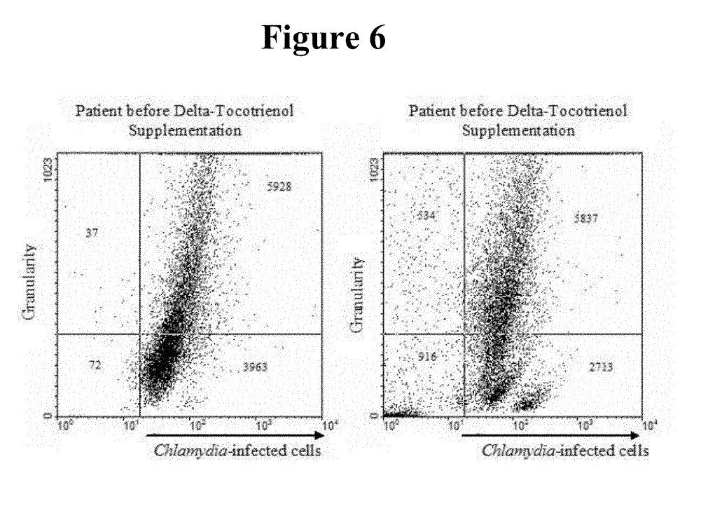

In this patient (FIG. 6), tocotrienol consumption reduced the granularity and amount of Chlamydia-infected cells, which is represented by the Chlamydia-negative population in the lower left quadrant of the panel.

Since flow cytometry samples a larger cell population [˜10,000 cells], this human study further supported and corroborated the chlamydial inhibition observed in the microscopic studies of Chlamydia infected cells treated in vitro (FIGS. 3 and 4) and shown in the earlier four examples.

This study shows that tocotrienol is effective in humans against Chlamydia as represented in circulating WBCs, including neutrophils, monocytes, lymphocytes, eosinophils, and basophils.

### Example 6

TABLE 2
Comparison of LDL Levels and Chlamydial Infection Status
in Hyperlipidemic Patients with Delta Tocotrienol Supplementation

| | Patient 1 | Patient 2 | Patient 3 |
|---|---|---|---|
| % Decrease in LDL | 20% | 7% | 25% |
| Chlamydial Infection Status | negative | positive | strong positive |
| before Delta-Tocotrienol | | | |
| Supplementation | | | |
| Chlamydial Infection Status | negative | negative | negative |
| after Delta-Tocotrienol | | | |
| Supplementation | | | |

Detection of Chlamydia-Infected White Blood Cells (WBC):

Smears of whole blood from each 10 ml hyperlipidemic HP blood sample, average donor age 54 years, were fixed with 70% MEOH for subsequent staining. Immunostaining used a 1:125 dilution of rabbit polyclonal anti-Chlamydia EB antibody followed by a 1:125 dilution of TRITC-conjugated goat anti-rabbit antibody (H+L) (Jackson ImmunoResearch, West Grove, Pa.). Slides were incubated at room temperature for 1 hour with each diluted antibody, rinsed, mounted and sealed as described above. Digital images of optical sections through these samples were acquired with a Zeiss LSM 510 Meta Confocal System.

In a clinical study, buffy coats from blood samples drawn both before and after delta-tocotrienol supplementation were immunostained for detection of Chlamydia-infected cells. The samples were tested for LDL levels both before and after delta-tocotrienol supplementation.

Tocotrienol lowered cholesterol in patients with or without prior chlamydial infection. Apparently, a cholesterol drop was more significant when the hyperlipidemic patient was Chlamydia-negative (Patient 1 vs. 2). As discussed earlier (Sawayama et al., 2003), pravastatin only lowered cholesterol marginally in Chlamydia-positive patients albeit that the carotid plaques were significantly reduced. This is consistent with the four-year clinical study described earlier, wherein patients who took tocotrienols had progressive carotid arteriosclerosis regression, but their cholesterol did not drop until the fourth year (Kooyenga et al., 2001).

However, in patient 2, the chlamydial infection status reversed from positive to negative with tocotrienol supplementation. When chlamydial inhibition responded to tocotrienol supplementation strongly (Patient 3), the cholesterol drop was also larger, similar to the patient that was Chlamydia-negative. Since tocotrienol is known to lower cholesterol, such cholesterol synthesis restriction inhibited the cholesterol-requiring growth of Chlamydia (Carabeo, 2003), seen in patients 2 and 3.

Tocotrienol supplementation reduced the cholesterol level and chlamydial infection in hyperlipidemic patients, and rendered the chlamydial infection status negative.

### Example 7

A corollary of example 6 is that any agent that restricts cholesterol synthesis would therefore inhibit or retard Chlamydia from hijacking cholesterol. Such agents include statins (e.g., lovastatin, simvastatin, pravastatin), citrus bioflavonoids, or specifically polymethoxylated flavones (e.g., tangeretin, nobiletin, hesperidin, rutin; English, 2004), polyphenolics (e.g., EGCG, catechins, resveratrol; Yamazaki, 2005), and an assortment of nutritional supplements (e.g., plant sterols, B vitamins, omega 3 fatty acids, insoluble and soluble fibers, red yeast rice; Strum, Faloon, 2005). This list is not meant to be limiting, but to exemplify the effect of cholesterol-lowering agents on restriction of Chlamydia infection. Other non-limiting examples for cholesterol-lowering, lipid-lowering, and cardiovascular support agents have recently been widely published (English, 2005; Granato, 2003 & 2005; Myers, 2006; Naguib, 2004; Srejic, 2006).

This study shows that any agent that restricts cholesterol/lipids also restricts Chlamydia infection/growth. In other words, any agent that restricts cholesterol restricts Chlamydia.

### Example 8

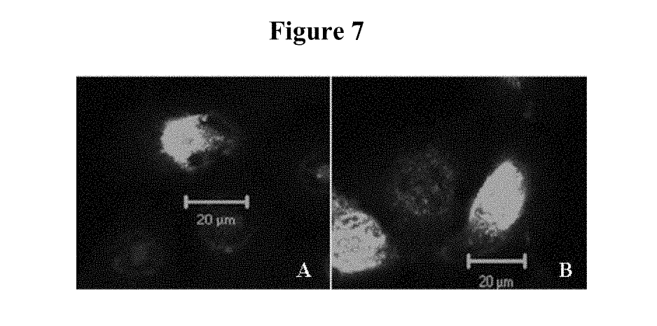

The methods of Example 1 were used in this study. Untreated cancer cells showed evidence of solid large inclusion (>20 μm; FIG. 7B) compared to intermediate size inclusions (20 μm) in non-cancerous murine macrophages (FIG. 4B) and even smaller inclusions in tocotrienol treated cells, both murine macrophages (FIG. 4A) and mammary tumor cells (10-15 μm; FIG. 7A).

The cholesterol content of cancer cells is very high because the rate-limiting HMG-CoA reductase enzyme for cholesterol synthesis in these cells is aberrantly elevated and the enzyme is resistant to sterol feedback regulation. Unequivocally, tocotrienol has been shown to inhibit the reductase synthesis and accelerate reductase degradation (Mo and Elson, 2004).

Since cancer cells are high in cholesterol content, they are more susceptible to infection by cholesterol-hijacking Chlamydia, which explains the engorged inclusions within the cells.

Tocotrienols are known to induce apoptosis of cancer cells. Contrarian to this, Chlamydia has been shown to inhibit the apoptosis of infected cells. Chlamydia-infected, tocotrienol-treated cancer cells in this example did not appear to become apoptotic. Although tocotrienol did not cause infected host cells to undergo apoptosis, it nonetheless disrupted the development of full-blown inclusions by the infecting Chlamydia. However, apoptosis with tocotrienol is expected with continual usage (Example 3), whereby the chlamydial EBs are eliminated with time (Example 1), as it has been numerously shown that control of Chlamydia is a process of controlling its progression (all earlier examples).

This study shows tocotrienol will arrest opportunistic chlamydial infections in cancer patients.

### Example 9

Chlamydial infection modulated activation of T-lymphocytes and was linked to a decrease in the antigen-presenting dendritic cell population in the human body. Therefore, the dendritic cells are inhibited from activating the T-lymphocytes, and the immunity of the individual against Chlamydia and other opportunistic pathogens is decreased. This is further supported by the implication of Chlamydia in autoimmune disease. Tocotrienol is known to boost the immune system to ward off viruses and bacterial infections, and has even been shown to assist T-lymphocytes in slowing down the progression of AIDS and increasing specific immune markers. Thus, tocotrienol also aids the immune system through the activation of dendritic cells and T-lymphocytes to fight off chlamydial infections.

This study shows tocotrienol will ward off pathogenic infections including Chlamydia via activation of antigen-presenting dendritic cells and T-lymphocytes.

This invention also applies to activation of antigen-presenting dendritic cells and T-lymphocytes against tumors and cancers.

### Example 10

Tocotrienol decreases high blood pressure in hypertensive rats. Primary pulmonary hypertensive (PPH) cells have enhanced expression of caveolae, which contributes to the elevated [Ca²⁺] associated with hypertension, and when treated with cholesterol-reducing agents such as statin, caveolae expression in these cells was modified and vasoconstriction was reduced. Since PPH cells have increased caveolae expression in cell membranes and Chlamydia entry into host cells involves lipid rafts, hypertensive patients are more susceptible to opportunistic chlamydial infections. Tocotrienol, like other cholesterol-reducing agents, decreases caveolae expression in cell membranes, downregulates the [Ca²⁺] in PPH cells, and protects against chlamydial infections.

This invention provides the application that tocotrienol fights off opportunistic chlamydial infection of hypertensive patients, and thereby reduces their blood pressure.

### Example 11

Metabolic syndrome is a cluster of cardiovascular risk factors, including elevated waist circumference, elevated triglycerides, reduced high-density lipoprotein cholesterol, elevated blood pressure, and elevated fasting glucose associated with obesity, and elevates one's risk of developing diabetes. Chlamydia is not a causative agent of diabetes, but seroactive chlamydial infections in diabetic patients are more frequent than in non-diabetic patients, meaning that diabetic patients may be more susceptible to infection by Chlamydia. Tocotrienol reduces the cardiovascular risk factor associated with metabolic syndrome, obesity, and diabetes, and therefore may reduce opportunistic chlamydial infections due to these factors.

This invention provides the application that tocotrienol reduces cardiovascular risk factors associated with metabolic syndrome, and thereby reduces the risk of diabetes and concomitant or subsequent infection by Chlamydia.

### Example 12

The mode of application of tocotrienols is important because Chlamydia is involved in many public health-related diseases. A non-limiting summary of these diseases is described in the background section. The mode of application of tocotrienol may be in form of aerosol sprays (to reach respiratory tract airways), oral ingestion via softgels, tablets or capsules (to reach vascular-organistic systems), topical creams, douches, and lotions (to reach genital sites), and topical liquid drops (to reach ocular sites).

Dosages: In one embodiment, the invention is drawn to a method comprising administering delta-T3 in a range from 10 to 1000 mg per day. In a preferred embodiment, the invention is drawn to a method comprising administering delta-T3 in a range from 20 to 500 mg per day. In a more preferred embodiment, the invention is drawn to a method comprising administering delta-T3 in a range from 50 to 150 mg per day. Treatment would be continuous with the delta-T3 being administered daily at the above-mentioned dosages (for as short as one month, preferably six months, and most preferably one year), or until infection and inflammation due to Chlamydia is cleared (e.g., no inclusion-forming units can be found in the patient's whole blood). However, for inhibition of chlamydial infection the dosage can be on the lower end (e.g., 1-100 mg/day), and the dosage duration can be indefinite, and begun before the subject has any evidence of infection by Chlamydia.

To reach respiratory tract infections, tocotrienol may be used in form of aerosol spray, at a dosage of 1-4 sprays per day at the above-mentioned dosages. For genital tract infections, tocotrienol may be administered in form of topical creams, lotions, or douches with 1-4 applications per day at the above-mentioned dosages. Vascular-organistic systems inflamed due to chlamydial infection can be treated with oral ingestion of tocotrienol with 1-4 softgels, tablets, or capsules per at the above-mentioned dosages. To treat ocular infections, including conjunctivitis, tocotrienol may be administered in form of liquid eye drops with 1-4 applications per day at the above-mentioned dosages.

1-19. (canceled)

20. A method of treatment for a Chlamydia infection, consisting of administering daily for at least 3 days a pharmaceutically effective amount of a tocotrienol to a mammal in need of treatment;
wherein the amount of the tocotrienol is a dose between 10 mg and 1000 mg per day, and
wherein the tocotrienol treats the Chlamydia bacterium.

21. The method of claim 20, where the treatment inhibits the developmental cell cycle of the Chlamydia bacterium.

22. The method of claim 20, where the treatment disrupts the developmental cell cycle of the Chlamydia bacterium.

23. The method of claim 20, where the tocotrienol is selected from the group consisting of a natural tocotrienol and a synthetic tocotrienol.

24. The method of claim 23, where the tocotrienol is an isomer of a tocotrienol.

25. The method of claim 24, where the isomer of tocotrienol is selected from the group consisting of alpha-tocotrienol, beta-tocotrienol, gamma-tocotrienol, delta-tocotrienol, desmethyl-tocotrienol, and didesmethyl-tocotrienol.

26. The method of claim 25, where the isomer of tocotrienol is delta-tocotrienol.

27. The method of claim 20, where the Chlamydia bacterium is selected from the group consisting of Chlamydia trachomatis, Chlamydia suis, Chlamydia muridarum, Chlamydiophila pneumoniae, Chlamydiophila psittaci, Chlamydiophila pecorum, Chlamydiophila abortis, Chlamydiophila felis, and Chlamydiophila caviae.

28. The method of claim 20, where mode of application of the tocotrienol is selected from the group consisting of aerosol spray, oral ingestion, creams, douches, lotions, and eye drops.

29. The method of claim 20, wherein the amount of the tocotrienol is a dose between 20 mg and 500 mg per day.

30. The method of claim 29, wherein the amount of the tocotrienol is a dose between 50 mg and 150 mg per day.

31. A method of treatment for a Chlamydia infection, consisting of administering daily for at least one month a pharmaceutically effective amount of a combination of a tocotrienol and an agent that restricts cholesterol to a mammal in need of treatment for a Chlamydia bacterium.

32. The method of claim 31, where the agent that restricts cholesterol is selected from the group consisting of a statin, a bioflavonoid, a polyphenolic, a polymethoxylated flavone, a plant sterol, a oryzanol, a policosanol, a B vitamin, CoQ10, an omega 3 fatty acid, a lecithin, garlic, a gugul lipids, an insoluble fiber, a soluble fiber, a soy protein, a chitosan, a red yeast rice, and a mineral.

33. A method of treatment for a Chlamydia infection, consisting of administering daily for at least 3 days a pharmaceutically effective amount of a tocotrienol to a mammal in need of treatment and at least one selected from the group consisting of an anti-inflammatory agent, am antioxidant agent, a COX inhibitor agent and a geranyl geraniol;
wherein the amount of the tocotrienol is a dose between 10 mg and 1000 mg per day, and
wherein the tocotrienol treats the Chlamydia bacterium.
